(19) United States
(12) Patent Application Publication
(10) Pub. No.: US 2024/0339203 A1
(43) Pub. Date: Oct. 10, 2024

(54) METHOD FOR ADJUSTING LIGHTING IN A HOSPITAL ROOM

(71) Applicant: CLINOMIC GMBH, Aachen (DE)

(72) Inventors: Arne PEINE, Aachen (DE); Lukas MARTIN, Aachen (DE)

(21) Appl. No.: 18/294,815

(22) PCT Filed: Aug. 4, 2022
(86) PCT No.: PCT/EP2022/000071

§ 371 Date: Feb. 2, 2024

(30) Foreign Application Priority Data
Aug. 5, 2021 (DE) 10 2021 120 439.9

(51) Int. Cl. G16H 40/20; G16H 10/60

(52) U.S. Cl. CPC G16H 40/20; G16H 10/60

(57) ABSTRACT

A method for adjusting a lighting in a hospital room, such as in an intensive care unit. The method comprises recording patient information specific to a medical condition of a patient in the hospital roomy; and activating of a lighting function of at least one lighting device in the hospital room depending on the recorded patient information, whereby the lighting function comprises a wake-up function and a day-night simulation.

The present invention relates to a method for adjusting lighting in a hospital room, in particular in an intensive care unit. The invention also relates to a system for adjusting lighting in a hospital room as well as to a computer programme.

It is known from the prior art, for example from publication U.S. Pat. No. 10,064,255 B2, that sensors can be used to detect the presence of an object or a person and, based on this, to control the lighting conditions in a room. Such detection can be used, for example, to recognise the presence of medical staff in a hospital room, which in particular indicates a situation in which an increased lighting level is required.

In particular, publication TW 2010 11943 A discloses an lighting system for mapping the colour spectrum of the course of the day. The same, in other words, is disclosed in publication CN 105 612 816 A.

In particular, publication CN 209 475 343 U discloses a wake-up system for patients coming out of a coma.

Publication CN 108 427 460 A discloses a system for monitoring environmental quality in hospitals. The system comprises in particular an air monitoring module with a temperature sensor, a humidity sensor, a sensor for the concentration of volatile organic substances, a sensor for the CO2 concentration and a sensor for the concentration of suspended particles in the air.

Publication CN 106 354 172 A discloses temperature control in hospitals.

Publication EP 3 335 632 A1 discloses a device for the combined detection of several signs of life such as respiratory activity, heartbeat activity and monitoring of the patient's movement. The publication further reveals that movement monitoring is of interest, for example, for patients in the recovery room from anaesthesia or for patients in a coma.

A disadvantage of the known solutions is that specific support for waking patients up is only possible to a limited extent. Also, the indication of presence and environmental monitoring is often cumbersome or, in many situations, not recognisable at all. Moreover, monitoring is too unreliable in many cases, i.e. for instance, frequently false detections occur. The indication of the ambient quality in publication CN 108 427 460 A is displayed in particular via a monitoring terminal, which can be more error-prone or involve greater technical effort, and also requires a longer read-off time. Another problem is that it is no longer possible to read it from a long distance away. In the publication EP 3 335 632 A1, no action takes place based on the detection of a movement, so that the monitoring of the patient can only be used to a limited extent.

It is therefore an object of the present invention to at least partially eliminate the disadvantages described above. In particular, it is the purpose of the present invention to provide improved support when patients wake up. Moreover, it is particularly a function to provide a technical aid for improving the treatment of a patient in a hospital.

The above mentioned function is solved by a method with the features of claim 1, a system with the features of claim 17 and by a computer programme with the features of claim 25. Further features and details of the invention can be seen from the respective sub-claims, the description and the drawings. Features and details described in connection with the method according to the invention naturally also apply in connection with the system used in accordance with the invention and the computer programme used in accordance with the invention, and vice versa in each case, so that reference is or can always be made to the individual aspects of the invention with respect to the disclosure.

The problem is solved in particular by a method for adjusting lighting in a hospital room, in particular in an intensive care unit, comprising:
recording patient information specific to a medical condition of a patient in the hospital room,
activating a lighting function of at least one lighting device in the hospital room depending on the recorded patient information, whereby the lighting function includes a wake-up function and a day-night simulation.

The preceding steps can be conducted one after the other or in any order, whereby individual steps can also be conducted repeatedly.

The following terminology should also be explained here:

In the context of the invention, indefinite and definite articles or numerals, e.g. “one”, “two”, “etc.”, are always to be understood as “at least” indications, unless expressly stated otherwise. Moreover, numerical data and data on process parameters and/or device parameters are to be understood in the technical sense, i.e. as being provided with the usual tolerances. Even from the explicit specification of “leastwise” or “at least” or similar, it should not be concluded that the simple use of the article or the number, i.e. without specifying “at least” or similar, a restriction, e.g. in the sense of “precisely”, should be implied. Specifically, the lighting device can also be several lighting devices, the time can also be several times, the interface can also be several interfaces, the sensor can also be several sensors, and the fixing means can also be several fixing means.

An adaptation is understood in particular to mean an adaptation in the sense of a control. A control is understood in particular to mean that incoming information is first forwarded to a control device. The incoming information can thereby be dependent on an evaluation of patient information, in particular medical data. Patient information is understood in particular as a selection of patient data, whereby patient data is understood in particular as all personal information of a patient that is recorded, processed and archived in a medical institution. The medical condition refers in particular to medically relevant physical or psychological occurrences, circumstances and changes in a patient. The intention is, for example, that the patient information comprises a heartbeat or an oxygen saturation of the patient or a time of anaesthesia and that a corresponding evaluation of the patient information leads to a triggering of a control of the lighting. The incoming information can then be processed in the control unit using a stored calculation algorithm and issued as an action instruction. The handling instructions here correspond in particular to the manipulation of the lighting provided inside and/or outside the hospital room. The term lighting refers in particular to at least one lighting device, for example a lamp, whereby also several lighting devices can also be arranged in a functionally and/or spatially coherent manner.

A hospital room refers in particular to a room in which patients are accommodated during their inpatient medical treatment in a hospital.

A lighting function is understood to mean in particular a special manipulation of the lighting so that it can for example vary in intensity and/or colour over time. The lighting function can thus implement a wake-up function, in which the brightness is gradually increased, in particular over a set period of time, preferably in order to support a patient in waking up. Furthermore, a day-night simulation can be implemented, with the lighting being switched on or off and/or a step-by-step variation of the lighting intensity being conducted automatically, in particular based on manually or automatically set times. The day-night simulation may include a simulation of natural day and night light and/or brightness according to the daily cycle and/or a simulation of sunrise and sunset. Furthermore, the day-night simulation can also adjust the lighting in such a way that the natural day and night light is simulated not outdoors but indoors (i.e. darkened by walls/ceiling).

It may further be possible for the method to further include:
determining patient data, in particular medical patient data, whereby the patient data is entered manually by an operator or is automatically retrieved from a patient data management system,
   whereby the recorded patient information at least partially includes the determined patient data, whereby activating the lighting function further comprises:
conducting an evaluation of the recorded patient information,
Activating the lighting function of at least one lighting device in the hospital room depending on a result of the evaluation of the recorded patient information.

It may be possible that a finding is partially entered by an operator, whereby the input in particular only contains the information relevant to adjusting the lighting. Alternatively, the information that is relevant for the adjusting of the lighting can be defined in advance in order to then retrieve it for the patient from the patient data management system. A classification of the patient's medical condition can be made, for example to determine a preferred duration of the patient's sleep. Furthermore, the age of the patient can be relevant for the adjusting of the lighting. It is also conceivable that an individual adjustment of the lighting, for example the duration and/or the intensity and/or the colour, can be adjusted in consultation with the patient. It may be possible that the lighting can be adjusted by the patient himself.

It is further possible that the lighting function includes a sleep therapy function. For instance, this can be configured using the patient data and/or manually so that sleep therapy, e.g. a certain sleep rhythm, is promoted and/or influenced by the lighting. Depending on the configuration, for example, the lighting can promote sleep by dimming the lighting and wake up by brightening the lighting.

It is also conceivable that the lighting function includes a wake-up function. To do this, the lighting can be increased at a pre-set time or when necessary (e.g. initiated by hospital staff) to wake the patient.

In addition, the lighting function may include an awake function in which the lighting is brightened (i.e. increased) when the patient has woken up. For example, sensors can be used to automatically detect the patient's awake state as patient information in order to activate this awake function based on the patient's awake state.

A further advantage may be that the acquired patient information comprises information about an anaesthesia of the patient, wherein the activation of the lighting function of the lighting device is performed based on the information about the anaesthesia, wherein the information about the anaesthesia comprises in particular information about an anaesthetic agent and/or an administered amount and/or a time of an administration of the anaesthetic agent. This can have the advantage that the patient is supported by the lighting function when waking up from anaesthesia. The lighting intensity can be increased gradually over a set period of time. Information about an anaesthetic agent and/or an administered quantity and/or a time of administration of the anaesthetic agent can be of relevance for the start time and the length of the defined period. It is also conceivable that a patient can be supported in the same way when waking up from a coma.

Pursuant to a further advantage, there may be a possibility that the method may further comprise:
conducting monitoring of the patient's medical condition,
conducting an assessment of the monitored condition, in particular by comparing it with the recorded patient information,
   whereby the activation of the lighting function of at least one lighting device takes place as a function of a result of the assessment carried out, in particular in order to visually indicate a critical state of the patient and/or a change of state, in particular an awakening, by lighting the lighting device. To put it simply, the medical condition of the patient is monitored, whereby various parameters such as heart rate, movement or oxygen saturation are conceivable for monitoring. Subsequently, in the event of a critical deviation or a deviation of relevance to medical personnel in one or more of these parameters, the medical personnel are then notified. Optionally, an emergency colour and/or lighting (possibly also lighting frequency such as flashing) can be set.

According to an advantage of the invention, the method may further comprise:
Conducting an activation of at least one external lighting device outside the hospital room as a function of the result of the assessment carried out in order to visually indicate a critical condition of the patient and/or a change of condition, in particular a wake-up, by means of a lighting of the external lighting device, wherein an lighting colour of the external lighting device is preferably determined for this purpose on the basis of the result of the assessment conducted.

This can have the advantage that the patient's condition can be assessed outside the hospital room, for example in a hospital corridor, or a potentially critical condition can be recognized.

In addition, it can be advantageous within the scope of the invention that the external lighting device is designed for lighting with at least two colours or as a segment display and the method further comprises:
conducting of a coded signalling of the result of the assessment by colour or via the segment display.

This can have the advantage that a more differentiated information disclosure of the result of the assessment is possible. The segments can be assigned different lighting colours. It may be conceivable that the segment display allows a display of numbers and/or letters to indicate different codes, where the codes may be specific to certain results of the assessment.

In a further possibility, it is possible that the method further comprises:
providing a notification interface to a notification system for notification from a medical staff,
conducting of the notification depending on the result of the performed assessment.

This can have the advantage that frequent controlling by medical staff become obsolete, so that medical staff have specifically more time for other activities. For example, a notification of a critical condition of the patient or a notification of the patient waking up is provided. Furthermore, it is conceivable that regular notification in the sense of monitoring occurs even if there is no change in the patient's condition.

In addition, within the scope of the invention it is conceivable that the method further comprises:
Activating the lighting function using at least one parameter of the lighting function, wherein the at least one parameter comprises at least one of the following: an intensity of the lighting, a colour of the lighting, a duration of the lighting, a frequency of the lighting, a lighting scheme.

In other words, the lighting function is defined in particular by various parameters, which can be stored in the sense of different lighting schemes. A lighting scheme can include a variation of the intensity of the lighting, colour, duration or frequency, whereby the variation can be time-dependent.

It is also advantageous when the process further comprises:
varying the parameter so that the wake-up function is provided when the recorded patient information corresponds to a first specification,
varying the parameter so that the day-night simulation is provided when the recorded patient information meets a second specification,
   whereby the wake-up function is preferably provided at least by increasing the lighting intensity over a predefined wake-up period and the day/night simulation is provided at least by adjusting the lighting intensity depending on the time of day.

A specification is understood to mean, in particular, a specific criterion. It is conceivable that the first specification corresponds to the condition of whether anaesthesia has been performed. For the second specification, it is conceivable, for example, that a certain day-night rhythm can be set for the patient without anaesthesia, which is adapted to his individual medical condition.

Optionally, It may be possible that the method further includes:
Providing an input device in order to activate the lighting function as a function of a user input at the input device, whereby at least one parameter for the lighting function, in particular a lighting intensity and/or a lighting duration and/or a lighting frequency and/or a lighting scheme, is defined by the user input. This can have the advantage that the user input can be individually coordinated to suit the patient. For example, the user's experience can be incorporated into the user input or a coordination can be carried out with the patient or their relatives. It may be possible that the input device is protected by a password or other identification, for example to only allow user input to medical personnel.

According to an advantageous further development of the invention, it may be provided that the day-night simulation is carried out automatically on the basis of at least one fixed time in order to provide time-controlled lighting by at least one lighting device, whereby at least one time corresponds to a current time or is determined by an operator or is determined automatically on the basis of patient information. The patient information can influence the execution of the day-night simulation due to the patient's medical condition or age.

Furthermore, it can be possible within the scope of the invention that the hospital room is designed to be windowless and/or shielded from daylight. This can have the advantage that the lighting is the only light source in the hospital room and can therefore be adjusted to provide the wake-up function or day-night simulation without interference, for example from sunlight.

Optionally, it can be possible that the recording of the patient information also includes:
determination of a movement of the patient, in particular a heart rate, the determination being carried out using a sensor.

By determining the movement, it is possible in particular to conclude whether the patient is sleeping and/or what kind of sleep phase he is currently in. The additional determination of the heart rate can enable a more differentiated analysis of the patient's sleep and medical condition.

It is also advantageous when the process also includes:
conducting of a detection of the presence of a medical staff, the detection being carried out in particular using a marker,
conducting an evaluation of the detection,
activation of at least one lighting function of at least one lighting device in the hospital room depending on the evaluation of the detection.

The presence is determined in particular by detecting a marker, for example a radio transmitter. If the presence of the person is determined during the evaluation of the detection, preferably with a predetermined identity, at least one lighting function of at least one lighting device in the hospital room is activated depending on the evaluation of the detection. This extension of the method can have the advantage that the brightness of a patient's room lighting device is automatically adjusted or deactivated, for the most part slightly, in order to create a more pleasant atmosphere for the patient and thus technically support the treatment.

Optionally, it is conceivable that the lighting can be adjusted via a radio connection. A WLAN, a Bluetooth or an NFC connection are particularly conceivable for a radio connection, although the list is not exhaustive. WLAN stands for Wireless Area Network and NFC stands for Near Field Communication.

A further advantage can be achieved within the scope of the invention if the lighting is adjusted via an electrical connection. An electrical connection can be made via a USB, a USB-C, a Micro-USB, or a Lightning interface, whereby the list is not exhaustive. USB stands for Universal Serial Bus.

The invention also relates to a system for adjusting lighting in a hospital room, in particular in an intensive care unit, exhibiting:
A recording device for detecting a patient information specific to a medical condition of a patient in the hospital room,
control device for activating a lighting function of at least one lighting device in the hospital room depending on the recorded patient information, whereby the lighting function includes a wake-up function and a day-night simulation.
   According to the invention, the system therefore brings with it the same advantages as have been described in detail with reference to the method pursuant to the invention.

On top of that, the system can be suitable to conduct the method pursuant to the invention.

A recording device is understood in particular to be a device which is suitable for recording the patient information. A computer is conceivable for this purpose, which has an interface to a patient data management system or an input device such as a keyboard.

A control device is understood in particular to mean a device which is designed to provide the lighting function of at least one lighting device in the hospital room depending on the recorded patient information, for example to reduce or increase the brightness of the lighting device. The term control here corresponds in particular to a directed influence on the performance of technical systems. A digital control system is provided in particular, whereby an analogue or binary control system may also be conceivable.

In a further possibility, it may be possible for the recording device to have at least one sensor, the sensor being designed in particular as a radar sensor.

Furthermore, it is conceivable that the recording device has at least one volume sensor in order to determine the medical condition or the alertness of the patient based on the sound level.

It is also conceivable that the recording device and the control device are connected to one another and are designed as a stand-alone system and, in particular, have a common housing.

Stand-alone means in particular that the system can fulfil its function without additional devices and is specifically designed as a modular construction. However, the system according to the invention can of course have interfaces for data exchange and/or for energy supply and/or for modular expansion.

Furthermore, it may be possible for the recording device and/or the control device to have at least one fastening means for fastening it under a hospital bed, the fastening means preferably being in the form of an adhesive.

It may also be envisaged that the devices have a common fastening means. It is also conceivable that the fastening means is designed for a screw, plug-in, latching or form-fitting connection to the hospital bed.

It is also conceivable that the recording device features a patient data interface to a patient data management system in order to provide patient data, in particular medical patient data, whereby the patient data interface is designed as an electrical or as a radio interface.

Within the scope of the invention, it may preferably be provided that the control device has a control interface in order to provide the adjustment of the lighting, wherein the control interface is designed as an electrical or radio interface.

It may optionally be possible for the system to comprise a notification interface to a notification system for notification of a medical staff member, the notification interface being designed as an electrical or radio interface.

The electrical interface can be designed as a USB, a USB-C, a Micro-USB, or a Lightning interface, although the list is not exhaustive. USB stands for Universal Serial Bus. The radio interface can be designed as a Bluetooth, a WLAN or an NFC interface, although the list is not exhaustive, with WLAN standing for Wireless Area Network and NFC for Near Field Communication.

Another object of the invention is a computer program comprising instructions which, when the computer program is executed by a computer, to prompt the computer to perform the following steps:
Initiating a recording of patient information specific to a medical condition of a patient in the hospital room,
Initiating activation of a lighting function of at least one lighting device in the hospital room depending on the recorded patient information, whereby the lighting function includes a wake-up function and a day-night simulation.

The computer program according to the invention thus brings with it the same advantages as have been described in detail with reference to the method according to the invention and the system according to the invention. In addition, the computer programme can be suitable to carry out the method according to the invention on the system according to the invention.

The computer programme is run, for example, by an electronic processing device for data processing, such as a processor and/or microcontroller, of the system according to the invention, which is electrically connected to the recording device and/or to the control device.

Further advantages, features and details of the invention emerge from the following description, in which exemplary embodiments of the invention are described in detail with reference to the drawings. In this respect, the features mentioned in the claims and in the description can each be essential to the invention individually or in any combination. It shows:

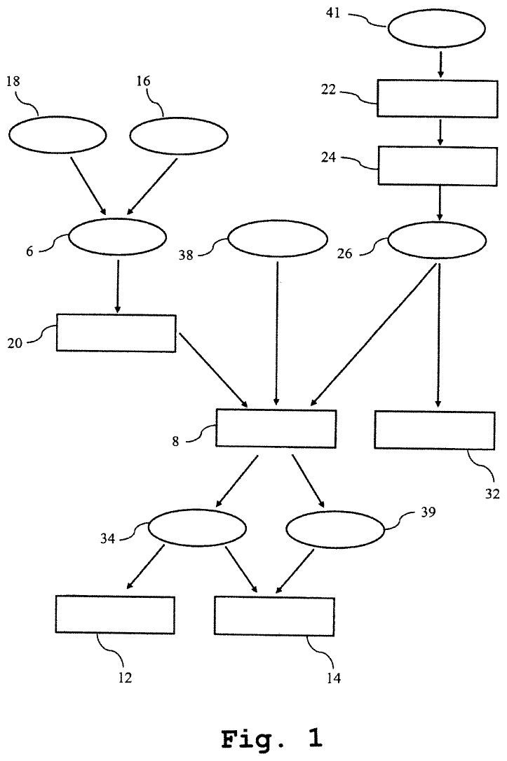

FIG. 1 An exemplary embodiment for a schematic sequence of the method according to the invention.

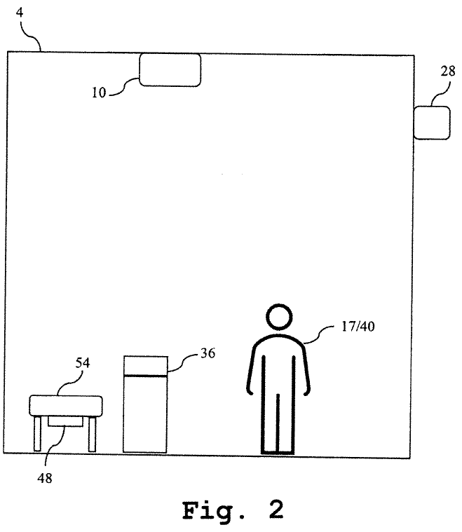

FIG. 2 A schematic arrangement of the system according to the invention in a hospital room.

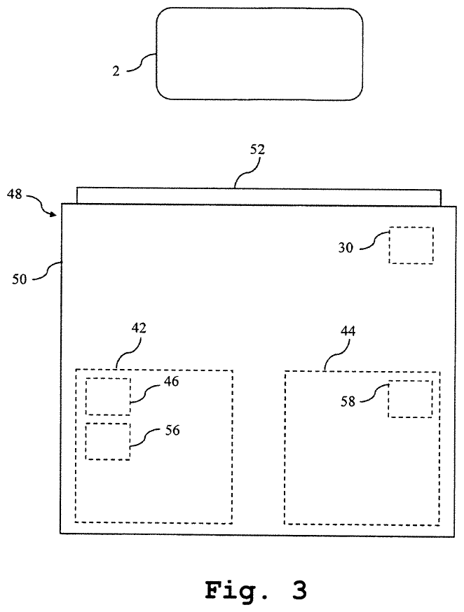

FIG. 3 A schematic representation of the structure of the system according to the invention and a lighting array.

FIG. 1 shows schematically a sequence of the method according to the invention. The lighting function 8 depends on three factors. The first factor is shown on the left-hand side, whereby the patient information 6 is determined here from the patient data 16 and/or information 18 about an anaesthetic, whereby it is then determined by the evaluation 20 whether and/or in what manner and/or to what extent the patient information 6 is taken into account for the lighting function 8. The second factor, the user input 38, is shown in the middle, which can have an influence on the lighting function 8. The right-hand side represents the third factor, whereby monitoring 22 of a movement 41 is carried out here. In the next step, an assessment 24 of the monitoring 22 follows in order to influence the lighting function 8, if necessary, by the result 26 of the assessment 24. In addition, the result 26 of the assessment 24 can lead to a notification 32, whereby in particular a medical personnel 40 is notified. The lighting function 8 then provides the functionalities of the wake-up function 12 and the day-night simulation 14. The wake-up function 12 depends on at least one parameter 34, such as a lighting intensity or duration. The day-night simulation 14 also depends on at least one parameter 34 and additionally on at least one time 39. At least one parameter 34 and at least one time 39 are determined or predetermined in particular by the lighting function 8.

FIG. 2 shows a schematic arrangement of the system 48 according to the invention in a hospital room 4. The system 48 is arranged under a hospital bed 54. Furthermore, a lighting device 10 in the hospital room 4 and an external lighting device 28 outside the hospital room 4 are visualized. Also shown is an operator 17, who may be a medical personnel 40. Furthermore, an input device 36 is arranged next to the sick bed 54.

FIG. 3 schematically illustrates the structure of a system 48 according to the invention and a lighting system 2. The system 48 has a housing 50 and a fastening element 52 on the top of the housing 50. A recording device 42, a control device 44 and a notification interface 30 are arranged in the housing 50. The recording device 42 further comprises a sensor 46 and a patient data interface 56. The control device 44 further comprises a control interface 58.

The above explanation of the embodiments describes the present invention solely in terms of examples. Of course, individual features of the embodiments can, if technically meaningful, be freely combined with one another without departing from the scope of the present invention.

### LIST OF REFERENCE SYMBOLS USED

2 lighting
4 hospital rooms
6 patient information
8 lighting function
10 lighting device
12 wake-up function
14 simulation
16 patient data
17 operator
18 information
20 evaluation
22 monitoring
24 assessment
26 result
28 external lighting device
30 notification interface
32 notification
34 parameter
36 input device
38 user input
39 time
40 staff
41 movement
42 recording device
44 control device
46 sensor
48 system
50 Housing
52 fastening elements
54 hospital bed
56 patient data interface
58 control interface

1. Method for adjusting lighting in a hospital room, comprising:
recording patient information which is specific to a medical condition of a patient in the hospital room,
activating a lighting function of at least one lighting device in the hospital room depending on the recorded patient information, whereby the lighting function includes a wake-up function and a day-night simulation.

2. The method according to claim 1, wherein the method further comprises:
determining patient data, whereby the patient data is entered manually by an operator or is automatically retrieved from a patient data management system,
whereby recorded patient information at least partially includes the determined patient data, whereby activating the lighting function further comprises:
conducting an evaluation of the recorded patient information,
activating the lighting function of at least one lighting device in the hospital room depending on a result of the evaluation of the recorded patient information.

3. Method according to claim 2, wherein the recorded patient information includes information about the patient's anaesthesia, whereby the activation of the lighting function of the lighting device is performed based on the information about the anaesthesia, whereby the information about the anaesthesia includes information about an anaesthetic and/or an administered amount and/or a time of administration of the anaesthetic.

4. Method according to claim 2, that wherein the method further comprises:
conducting a monitoring of the patient's medical condition,
conducting an assessment of the monitored condition, by comparing it with the recorded patient information,
wherein the activation of the lighting function of the at least one lighting device takes place depending on a result of the assessment carried out by lighting of the lighting device, to a critical level condition of the patient and/or to indicate a change in condition.

5. The method according to claim 4, wherein the method further comprises:
conducting an activation of at least one external lighting device outside the hospital room as a function of the result of the assessment carried out, in order to visually indicate a critical state of the patient and/or a change of state, by means of an lighting of the external lighting device, a lighting colour of the external lighting device being determined for this purpose on the basis of the result of the assessment carried out.

6. Method according to claim 5, wherein the external lighting device is designed for lighting with at least two colours or as a segment display and the method further comprises:
conducting of a coded signalling of the result of the assessment according to colours or via the segment display.

7. Method according to claim 4, wherein the method further comprises:
providing a notification interface to a notification system for a notification from medical staff,
conducting of the notification depending on the result of the performed assessment.

8. Method according to claim 1, wherein the method further comprises:
activating the lighting function based on at least one parameter of the lighting function, whereby at least one parameter comprises at least one of the following: an intensity of the lighting, colour of the lighting, a duration of the lighting, a frequency of the lighting, a lighting scheme.

9. The method according to claim 8, wherein the method further comprises:
varying the parameter so that the wake-up function is provided when the recorded patient information corresponds to a first specification,
varying the parameter so that the day-night simulation is provided when the recorded patient information corresponds to a second specification,
whereby the wake-up function is provided at least by a temporal increase in the lighting intensity over a predefined wake-up period and the day-night simulation is provided at least by a time-of-day-dependent adjustment of the lighting intensity.

10. Method according to claim 1, wherein the method further comprises:
providing an input device to activate the lighting function depending on a user input on the input device,
whereby at least one parameter for the lighting function is defined by the user input.

11. Method according to claim 1, wherein the day-night simulation is carried out automatically based on at least one fixed time in order to provide time-controlled lighting by at least one lighting device, whereby at least one time corresponding to a current time or is determined by an operator or is determined automatically based on the patient information.

12. Method according to claim 1, wherein the hospital room is designed to be windowless and/or shielded from daylight.

13. Method according to claim 1, wherein the recording of the patient information also includes:
determination of a movement of the patient, in the determination being carried out using a sensor.

14. Method according to claim 1, wherein the method further comprises:
conducting of a detection of the presence of a medical staff, the detection being carried out based on a marker,
conducting of an evaluation of the detection,
activation of at least one lighting function of at least one lighting device in the hospital room depending on the evaluation of the detection.

15. Method according to claim 1, wherein the lighting is adjusted via a radio connection.

16. Method according to claim 1, wherein the adjustment of the lighting is carried out via an electrical connection.

17. System for adjusting a lighting in a hospital room comprising:
a recording device for detecting patient information which is specific to a medical condition of a patient in the hospital room,
control device for activating a lighting function of at least one lighting device in the hospital room depending on the recorded patient information, whereby the lighting function includes a wake-up function and a day-night simulation.

18. System according to claim 17, wherein the recording device features at least one sensor, whereby the sensor is designed as a radar sensor.

19. System according to claim 17, wherein the recording device and the control device are connected to one another and are designed as a stand-alone system and have a common housing.

20. System according to claim 17, wherein the recording device and/or the control device features at least one fastening means in order to carry out fastening under a hospital bed, whereby the fastening means is designed as an adhesive.

21. System according to claim 17, wherein the recording device has a patient data interface to a patient data management system in order to provide patient data, whereby the patient data interface is designed as an electrical or as a radio interface.

22. System according to claim 17, wherein the control device features a control interface in order to provide the adjustment of the lighting, whereby the control interface is designed as an electrical or as a radio interface.

23. System according to claim 17, wherein the system comprises a notification interface to a notification system for a notification from a medical staff, whereby the notification interface is designed as an electrical or as a radio interface.

24. System according to claim 17, wherein the system is designed to carry out the following steps:
recording patient information which is specific to a medical condition of a patient in the hospital room,
activating a lighting function of at least one lighting device in the hospital room depending on the recorded patient information, whereby the lighting function includes a wake-up function and a day-night simulation.

25. Computer programme, comprising instructions which, that when the computer programme is executed by a computer, to cause it to carry out the following steps:
initiating of one recording of patient information which is specific to a medical condition of a patient in the hospital room,
initiating activation of a lighting function of at least one lighting device in the hospital room depending on the recorded patient information, where the lighting function includes a wake-up function and a day-night simulation.
